(19) United States
(12) Patent Application Publication
(10) Pub. No.: US 2006/0039961 A1
(43) Pub. Date: Feb. 23, 2006

(54) Percutaneous absorption accelerator

(76) Inventors: Satoshi Inaoka, Osaka City (JP); Shuichi Tsunetsugu, Kobe City (JP)

Correspondence Address: CHOATE, HALL & STEWART LLP, TWO INTERNATIONAL PLACE, BOSTON, MA, 02110, US

(21) Appl. No.: 11/137,883

(22) Filed: May 26, 2005

(30) Foreign Application Priority Data
May 28, 2004 (JP) 2004-160158
May 20, 2005 (CA) 2,508,096

(51) Int. Cl. A61K 31/205; A61K 9/70

(52) U.S. Cl. 424/449; 514/554

(57) ABSTRACT

The present invention has as an object to provide a composition for external use on skin whose percutaneous absorption of an active component is increased, whereby a large amount of the active component can be delivered into the skin. By employing diglycerol and a low molecular weight betaine together, a percutaneous absorption accelerator excellent in permeability of an active component is provided. According to the percutaneous absorption accelerator of the present invention, the accelerated percutaneous absorption amount of an active component is greatly increased, whereby an effect which the active component essentially possesses is sufficiently exhibited.

### BACKGROUND OF THE INVENTION

1. Field of the Invention

The present invention relates to a percutaneous absorption accelerator. In addition, the present invention relates to a composition for external use on skin comprising the percutaneous absorption accelerator and an active component.

2. Description of Related Art

A number of components such as a component having an excellent pharmacological action on living body and a component regulating a physiological action in living body are known. In the case where a formulation is designed by blending such an active component in a preparation for external use and allowing the active component to be absorbed into the skin thereby exhibiting the effect, percutaneous absorption of the active component is important. It is because any effective action cannot be exhibited without permeation of the active component into a biological barrier, namely skin. Therefore, means for enhancing the percutaneous absorption amount of an active component which exerts a pharmacological action or a physiological action on the living body, and for allowing the active component to be efficiently absorbed was needed.

With regard to a composition for external use on skin, a number of compounds enhancing percutaneous absorption of an active component are known. Examples of the compound include a polyhydric alcohol that enhances percutaneous absorption (Non-patent document 1: Journal of Controlled Release, 42, 1996, 217-228) and the like.

On the other hand, a low molecular weight betaine represented by trimethylglycine is known to have typically a humectant effect as the use in cosmetic products, and other than this, an antistatic effect, a bacteriostatic effect, effect on repairing hair damage and the like. In addition, it is known that a low molecular weight betaine enhances percutaneous absorption of a whitening component such as an ascorbic acid derivative. It is also known that, in a preparation for external use on skin containing a whitening agent, a low molecular weight betaine, and an oil component selected from silicone oils and plant oils, percutaneous absorption of a whitening agent is improved thereby enhancing an effect on improving skin pigmentation and dullness by the whitening agent, enhancing an effect on improving rough skin and humectant function and providing a good sensation in use as well as enhancing a whitening effect (Patent document 1: JP-A-2001-89321).

Diglycerol is known to have typically a humectant effect as the use in cosmetic products. However, the effect on enhancing percutaneous absorption was not known.

### SUMMARY OF THE INVENTION

The present invention provides a percutaneous absorption accelerator and a composition for external use on skin comprising the percutaneous absorption accelerator and an active component.

As a result of diligent research in order to overcome the problems described above, the present inventors discovered that, by employing diglycerol and a low molecular weight betaine together, percutaneous absorption amount of an active component could be dramatically increased.

That is, the present invention relates to a percutaneous absorption accelerator or a composition for external use on skin described in any of (1) to (3) shown in the following. (1) A percutaneous absorption accelerator comprising combination of diglycerol and a low molecular weight betaine. (2) The percutaneous absorption accelerator described in (1), wherein the low molecular weight betaine is trimethylglycine. (3) A composition for external use on skin, comprising a percutaneous absorption accelerator described in (1) or (2) and an active component.

### DETAILED DESCRIPTION OF THE INVENTION

In the specification of the present application, “%” means “% by weight” unless otherwise indicated.

According to the present invention, by employing diglycerol and a low molecular weight betaine together, a percutaneous absorption accelerator having a high effect can be provided. In a composition for external use on skin comprising the percutaneous absorption accelerator of the present invention and an active component, enhancement of the percutaneous absorption of the active component is dramatically increased, whereby an effect which the active component essentially possesses can be sufficiently exhibited.

The percutaneous absorption accelerator of the present invention comprises combination of diglycerol that is a known compound and a low molecular weight betaine. The low molecular weight betaine means one having a molecular weight of 200 or less, and forming an amphoteric ion in the molecule. Specific examples thereof include a quaternary ammonium base, a quaternary phosphonium base, a tertiary sulfonium base, and the like. They exhibit little surfactant activity. Among these, an N,N,N-trialkylamino acid represented by formula (1) shown below is preferable.

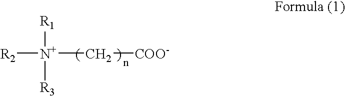

wherein R₁, R₂, and R₃ independently represent an alkyl group having 1 to 6 carbon atoms; and n represents 1 to 6.

As R₁ to R₃, a straight or branched chain alkyl group having 1 to 6 carbon atoms can be widely employed. That is, as examples thereof, mention may be made of, a methyl group, an ethyl group, a propyl group, an isopropyl group, a butyl group, an isobutyl group a sec-butyl group, tert-butyl group, a pentyl group, an isopentyl group, a neopentyl group, a tert-pentyl group, a hexyl group, an isohexyl group, a 3-methylpentyl group, 2,2-dimethylbutyl group, a 2,3-dimethylbutyl group, and the like. R₁ to R₃ may be the same or different.

In particular, in the case of n=1, examples thereof include trimethylglycine, triethylglycine, tripropylglycine, and triisopropylglycine; in the case of n=2, examples thereof include trimethyl-beta-alanine; and in the case of n=3, examples thereof include trimethyl-gamma-aminobutyric acid, and the like. Trimethylglycine is preferable.

In addition, the low molecular weight betaines described above may have substituents. In particular, in the case of n=1, as examples thereof, mention may be made of N,N,N-trimethylalanine, N,N,N-triethylalanine, N,N,N-triisopropylalanine, N,N,N-trimethylmethylalanine, carnitine, acetyl carnitine, and the like. Carnitine is preferable.

The percutaneous absorption accelerator of the present invention comprises combination of diglycerol and a low molecular weight betaine, and the blending ratio can be appropriately selected. However, the blending ratio of the low molecular weight betaine to 1 part by weight of diglycerol preferably ranges from 0.01 to 10 parts by weight, more preferably, from 0.1 to 7.parts by weight, and particularly preferably from 0.1 to 5 parts by weight.

The composition for external use on skin of the present invention comprises a percutaneous absorption preparation containing diglycerol and a low molecular weight betaine as the active components together with another active component. The blending amount of the percutaneous absorption preparation in the composition for external use on skin can be appropriately selected in view of the effects of the present invention. However, the blending amount thereof as the total amount of the contained diglycerol and low molecular weight betaine may preferably range from 1 to 90% by weight, more preferably from 5 to 60% by weight, and particularly preferably from 5 to 30% by weight.

By using the percutaneous absorption preparation containing diglycerol and a low molecular weight betaine as the active components, percutaneous absorption amount of the active component in the composition can be dramatically improved. Preferably, the active component is a negatively charged component, and it has a percutaneous absorption property that the effect can be sufficiently exhibited even if it is blended in an amount of, for example, 1% by weight or more in the composition for external use on skin. In view of the effects of the present invention, the blending amount of the active component can be appropriately selected depending on the type of the active component. However, in the composition for external use on skin containing the active component in an amount of commonly 0.01% by weight or higher, preferably 0.1% by weight or higher, more preferably 1% by weight or higher, particularly preferably 5% by weight or higher, the percutaneous absorption of the active component is increased, whereby a high effect can be exhibited. In addition, the active component is preferably a component with a relatively low molecular weight. In the case where the molecular weight is, for example, in the range of from 50 to 1000, more preferably from 50 to about 500, the active component can be percutaneously absorbed effectively.

As the active component in the composition for external use on skin of the present invention, in addition to components employed in the field of cosmetics such as a whitening component, an anti-inflammatory component, an antibacterial component, a cell stimulating component, an astringent component, an antioxidant component, a component for ameliorating acne, an anti-ageing component, a component for accelerating syntheses for biological ingredients such as collagen, a blood circulation accelerator component and a humectant component, components employed in the field of medicines and the like may be exemplified.

For example, as examples of the whitening component, mention may be made of ascorbic acid or derivatives thereof; arbutin; ellagic acid; phytic acid; rucinol; chamomile ET; vitamins such as vitamin A or derivatives thereof, vitamin E or derivatives thereof, pantothenic acid or derivatives thereof; and the like. Among these, as preferable examples thereof, mention may be made of pantothenic acid or derivatives thereof, ellagic acid, phytic acid, vitamin A or derivatives thereof, and vitamin E or derivatives thereof. The whitening components described above can be employed alone or in combination of two or more kinds thereof.

Plant components exhibiting whitening effects may be employed as whitening components. As examples of the plant components described above, mention may be made of components derived from plants such as iris, almond, aloe, gingko, oolong tea, rose hips, Scutellaria baicalensis, Coptis japonica, Hypericum erectum, dead nettle, seaweed, Pueraria lobata, cape jasmine, Sophora flavescens, chlorella, Schlechtendaria chinensis, wheat, rice, rice germ, oryzanol, rice bran, Asarum sieboldii, Zanthoxyli fructus, perilla, Paeoniae radix, Cnidium officinale, Morus australis, soybean, fermented soybean, tea, Angelica sinensis, Calendula officinalis, garlic, Hamamelis virginiana, safflower, Paeonia suffruticosa, Coix lacryma-jobi, Angelica sinensis [sic], Salvia leucantha, Uncaria gambir, asebiwarabi [phonetic spelling], Podocarpus macrophyllus, Flammulina velutipes, Diospyros kaki, Catalpa ovata, black bean, Gentiana amarella, Scrophularia buergeriana, Smilax medoca, snap bean, shokuma [phonetic spelling], Paris polyphylla, sage, Peuceadanum praeruptorum, Japanese radish, Ericaceae, Lespedeza homoloba, toshin [phonetic spelling], Picrasma quassioides, parsley, holly, hop, Lespedeza cyrtobotrya, clove, Glycyrrhiza glabra, and the like. Preferable are plant components derived from iris, aloe, gingko, oolong tea, rose hips, Scutellaria baicalensis, Coptis japonica, Hypericum erectum, dead nettle, seaweed, Pueraria lobata, cape jasmine, Sophora flavescens, Schlechtendaria chinensis, wheat, rice, rice bran, Asarum sieboldii, Zanthoxyli fructus, perilla, Paeoniae radix, Cnidium officinale, Morus australis, tea, Angelica sinensis, Calendula officinalis, Hamamelis virginiana, safflower, Paeonia suffruticosa, Coix lacryma-jobi, Salvia leucantha, Uncaria gambir, Flammulina velutipes, Diospyros kaki, Catalpa ovata, black bean, Gentiana amarella, Smilax medoca, snap bean, Paris polyphylla, sage, Peuceadanum praeruptorum, Japanese radish, Ericaceae, Lespedeza homoloba, toshin [phonetic spelling], Picrasma quassioides, parsley, holly, hop, clove, Glycyrrhiza glabra, and Angelica sinensis [sic]. More preferable are plant components derived from iris, aloe, gingko, rose hips, Scutellaria baicalensis, Coptis japonica, Hypericum erectum, cape jasmine, Sophora flavescens, rice, rice bran, Asarum sieboldii, Paeoniae radix, Cnidium officinale, Morus australis, tea, Angelica sinensis, Calendula officinalis, Hamamelis virginiana, safflower, Paeonia suffruticosa, Salvia leucantha, Uncaria gambir, Flammulina velutipes, Diospyros kaki, sage, Japanese radish, Ericaceae, parsley, hop, Glycyrrhiza glabra, and Coix lacryma-jobi. In the case of employing the plant components described above in the preparation for external use on skin of the present invention, the form of the plant components is not particularly limited. In general, the form such as a plant extract, an essential oil, or the like, can be employed.

As examples of anti-inflammatory components, mention may be made of allantoin, calamine, glycyrrhizic acid or derivatives thereof, glycyrrhetic acid or derivatives thereof, zinc oxide, guaiazulene, tocopherol acetate, pyridoxine hydrochloride, menthol, camphor, turpentine oil, indomethacin, salicylic acid or derivatives thereof, and the like. Preferable are allantoin, glycyrrhizic acid or derivatives thereof, glycyrrhetic acid or derivatives thereof, guaiazulene, and menthol.

As examples of antibacterial components, mention may be made of chlorhexidine, salicylic acid, benzalkonium chloride, acrinol, ethanol, benzethonium chloride, cresol, gluconic acid and derivatives thereof, povidone iodine, potassium iodide, iodine, isopropyl methylphenol, triclocarban, triclosan, sensitizing dye No. 101, sensitizing dye 201, paraben, phenoxyethanol, 1,2-pentane diol, alkyldiaminoglycine hydrochloride, and the like. As preferable examples thereof, mention may be made of benzalkonium chloride, benzethonium chloride, gluconic acid andderivativesthereof, isopropylmethylphenol, triclocarban, triclosan, sensitizing dye No. 101, sensitizing dye No. 201, paraben, phenoxyethanol, 1,2-pentane diol, alkyldiaminoglycine hydrochloride, and the like. More preferable are benzalkonium chloride, gluconic acid and derivatives thereof, benzethonium chloride, and isopropyl methylphenol.

As examples of cell stimulating components, mention may be made of amino acids such as γ-aminobutyric acid, ε-aminopuronic acid, and the like; vitamins such as retinol, thiamine, riboflavin, pyridoxine hydrochloride, pantothenic acid, and the like; alpha-hydroxylic acids such as glycolic acid, lactic acid, and the like; tannin, flavonoid, saponin, allatoin, sensitizing dye No. 301, and the like. Preferable are amino acids such as y-aminobutyric acid, ε-aminopuronic acid, and the like; and vitamins such as retinol, thiamine, riboflavin, pyridoxine hydrochloride, pantothenic acid, and the like.

As examples of astringent components, mention may be made of metal salts such as alum, chlorohydroxyaluminum, aluminum chloride, allantoin aluminum salt, zinc sulfate, aluminum potassium sulfate, and the like; and organic acids such as tannic acid, citric acid, lactic acid, succinic acid, and the like. Preferable are alum, chlorohydroxyaluminum, aluminum chloride, allantoin aluminum salt, aluminum potassium sulfate, and tannic acid.

As examples of antioxidant components, mention may be made of tocopherol and derivatives thereof, butylhydroxyanisole, dibutylhydroxytoluene, sodium hydrogen sulfite, erythorbic acid and salts thereof, flavonoid, glutathione, glutathione peroxidase, glutathione-S-transferase, catalase, superoxide dismutase, thioredoxin, taurine, thiotaurine, hypotaurine, and the like. Preferable are tocopherol and derivatives thereof, thiotaurine, hypotaurine, thioredoxin, and flavonoid.

As examples of anti-ageing components, mention may be made of retinoid (retinol, retinoic acid, retinal, and the like), pangamic acid, kinetin, ursolic acid, an extract of Curcuma longa, sphingosine derivatives, silicon, silicic acid, N-methyl-L-serine, mevalonolactone, and the like. Preferable are retinoid (retinol, retinoic acid, retinal, and the like), and kinetin.

As examples of humectant components, mention may be made of amino acids and derivatives thereof such as alanine, serine, leucine, isoleucine, threonine, glycine, proline, hydroxyproline, glucosamine, theanine, and the like; peptides such as collagen, collagen peptide, gelatin, and the like; polyhydric alcohols such as glycerol, 1,3-butylene glycol, propylene glycol, polyethylene glycol, and the like; sugar alcohols such as sorbitol and the like; phospholipids such as lecithin, hydrogenated lecithin, and the like; mucopolysaccharides such as hyaluronic acid, heparin, chondroitin, and the like; components based on NMF such as lactic acid, sodium pyrrolidone carbonate, urea, and the like; polyglutamic acid, and the like. Preferable are alanine, serine, glycine, proline, hydroxyproline, glucosamine, theanine, collagen, collagen peptide, glycerol, 1,3-butylene glycol, hydrogenated lecithin, hyaluronic acid, heparin, chondroitin, lactic acid, sodium pyrrolidone carbonate, and polyglutamic acid.

In the composition for external use on skin of the present invention, in addition to the components described above, surfactants, solubilizing components, fats and oils, sugars, or the like can be further blended.

As examples of surfactants employed herein, mention may be made of various nonionic surfactants, examples of which include polyoxyethylene (hereinafter, referred to as POE) branched alkyl ethers such as POE octyldodecyl alcohol, POE 2-decyltetradecyl alcohol, and the like; POE alkyl ethers such as POE oleyl alcohol ether, POE cetyl alcohol ether, and the like; sorbitan esters such as sorbitan monooleate, sorbitan monoisostearate, sorbitan monolaurate, and the like; POE sorbitan esters such as POE sorbitan monooleate, POE sorbitan monoisostearate, POE sorbitan monolaurate, and the like; glycerol fatty acid esters such as glycerol monooleate, glycerol monostearate, glycerol monomyristate, and the like; POE glycerol fatty acid esters such as POE glycerol monooleate, POE glycerol monostearate, POE glycerol monomyristate, and the like; POE hardened castor oil fatty acid esters such as POE dihydrocholesterol ester, POE hardened castor oil, POE hardened castor oil isostearate, and the like; POE alkyl aryl ethers such as POE octyl phenyl ether, and the like; glycerol alkyl ethers such as monoisostearyl glyceryl ether, monomyristyl glyceryl ether, and the like; POE glycerol alkyl ethers such as POE monostearyl glyceryl ether, POE monomyristyl glyceryl ether, and the like; polyglycerol fatty acid esters such as diglyceryl monostearate, decaglyceryl decastearate, decaglyceryl decaisostearate, diglyceryl diisostearate, and the like; or natural surfactants such as lecithin, hydrogenated lecithin, saponin, surfactin sodium salt, cholesterol, bile acid, and the like; and the like. The surfactants described above can be employed alone or in combination of two or more kinds thereof.

In the case of employing the surfactants, the ratio thereof blended in the composition for external use on skin of the present invention is not particularly limited as long as they do not provide adverse effects to the skin or mucosa and do not impair the effects of the present invention. They can be appropriately selected and employed in the ratio ranging from 0.01 to 30% by weight in the composition for external use on skin.

As fats and oils, they are not particularly limited as long as they are those employed as components of preparations for external use in the field of medicines, quasi drugs, or cosmetics. As examples thereof, mention may be made of synthetic oils such as middle-chain fatty acid triglyceride, and the like; vegetable oils such as soybean oil, rice oil, rapeseed oil, cotton seed oil, sesame oil, safflower oil, castor oil, olive oil, cacao butter, camellia oil, sunflower oil, palm oil, linseed oil, perilla oil, shea oil, saru [phonetic spelling] oil, coconut oil, Japan wax, jojoba oil, grape seed oil, avocado oil, and the like; animal oils such as mink oil, yolk oil, beef tallow, milk fat, lard, and the like; waxes such as beeswax, spermaceti wax, lanolin, carnauba wax, candelilla wax, and the like; hydrocarbons such as liquid paraffin, squalene, squalane, microcrystalline wax, ceresin wax, paraffin wax, vaseline, and the like; natural or synthetic fatty acids such as lauric acid, myristic acid, stearic acid, oleic acid, isostearic acid, behenic acid, and the like; natural or synthetic higher alcohols such as cetanol, stearyl alcohol, hexyldecanol, octyldecanol, lauryl alcohol, and the like; esters or ethers such as isopropyl myristate, isopropyl palmitate, octyldodecyl myristate, octyldodecyl oleate, cholesterol oleate, and the like; silicone oils; and the like. The fats and oils described above can be employed alone or in combination of two or more kinds thereof.

In the case of employing the fats and oils described above, with regard to the ratio thereof blended in the composition for external use on skin of the present invention, they can be appropriately selected and employed in the ratio ranging from 0.01 to 70% by weight in the composition for external use on skin.

As sugars, they are not particularly limited as long as they are those employed as components of preparations for external use in the field of medicines, quasi drugs, or cosmetics. As examples thereof, mention may be made of monosaccharides (such as glucose, galactose, mannose, ribose, arabinose, xylose, deoxyribose, fructose, ribulose, lyxose, and the like), disaccharides (such as sucrose, trehalose, lactose, maltose, cellobiose, and the like), oligosaccharides (such as lactulose, raffinose, pullulan, and the like), cellulose and derivatives thereof (such as methylcellulose, ethylcellulose, hydroxyethylcellulose, hydroxypropylcellulose, carboxymethylcellulose, carboxyethylcellulose, nitrocellulose, and the like), polymer sugars (such as chondroitin sulfate, hyaluronic acid, dermatan, heparan, heparin, keratan, and salts thereof (pharmaceutically or physiologically acceptable salts such as sodium chondroitin sulfate, sodium hyaluronate, dermatan sulfate, haparan sulfate, keratan sulfate, and the like), and the like), and sugar alcohols (such as mannitol, xylitol, erythritol, pentaerythritol, maltitol, sorbitol, polydextrose, and the like), and in addition, xylose, inositol, dextrin and derivatives thereof, honey, a muscovado extract, and the like. The sugars described above may be employed alone or in combination of two or more kinds thereof.

In the composition forexternal use on skin of the present invention, various components which are generally employed as components of preparations for external use in the field of medicines, quasi drugs, or cosmetics, such as amino acids, reducers for irritation, thickening agents, preservatives, UV controlling agents, coloring agents, dispersing agents, pH adjustors, perfumes, and the like can be further blended if necessary. The components described above can be freely employed alone or in combination of two or more kinds thereof.

The composition for external use on skin of the present invention can be prepared in a preferable form of a paste, a mousse, a gel, a liquid, a milky lotion, a cream, a sheet (base material carrier), an aerosol, a spray, or the like. They can be produced in a conventional method known in the art.

The composition for external use on skin of the present invention can be formed into various compositions in the field of cosmetics, medicines for external use, or quasi drugs for external use, including compositions for scalp such as preparations for promoting hair growth and hair restoration preparations, makeup cosmetics such as foundations, lipsticks, mascaras, eye shadows, eyeliners, eyebrow colors and nail varnishes; base cosmetics such as milky lotions, creams, lotions, oils and facial packs; cleansing compositions such as face cleansing compositions, cleansers and body washes; underarm deodorants, athlete's foot remedies, anti-itching preparations, wound healing preparations, dry bathing preparations, cleaning preparations, anti-inflammatory analgesic preparations, acne remedies, hemorrhoidal preparations, sterilizing preparations, whitening preparations, UV controlling preparations, and the like.

### EXAMPLE

In the following, the present invention is described in detail based on Examples and Test Examples. It should be understood that the present invention is not limited to the Examples and the like. In each of the composition examples described below, “%” means % by weight (W/W), unless otherwise indicated.

### Test Example 1

### Percutaneous Absorption Test

The dorsal skin of a hairless mouse was pinched and fixed between percutaneous absorption cells, and the cell at the receptor side was filled with saline and the cell at the donor side was filled with each of the preparations shown in Table 1. The solutions in both cells were incubated at 37° C. while being agitated with a stirrer bar. After 24 hours, the concentration of ascorbic acid in the solution at the receptor side was measured by high performance liquid chromatography. The measurement results are shown in Table 1. The results are represented by the relative ratio based on the permeation amount of ascorbic acid in Comparative Example 1 (solution containing only ascorbic acid), which was defined as 1.

TABLE 1

| | Comparative | Comparative | Comparative | | |
|---|---|---|---|---|---|
| | Example 1 | Example 2 | Example 3 | Example 1 | Example 2 |
| L-ascorbic acid | 10% | 10% | 10% | 10% | 10% |
| Diglycerol | — | 5% | — | 5% | 10% |
| Trimethylglycine | — | — | 5% | 5% | 2% |
| Water | 90% | 85% | 85% | 80% | 78% |
| Relative ratio of | 1.0 | 1.2 | 1.5 | 2.0 | 1.8 |
| percutaneous | | | | | |
| permeation amount | | | | | |

From the results of the test, it could be confirmed that, in the case of combining diglycerol and trimethylglycine, the permeation amount of ascorbic acid, namely, percutaneous absorption amount thereof was increased in multiples. The composition for external use on skin comprising ascorbic acid and a percutaneous absorption accelerator comprising combination of diglycerol and trimethylglycine can deliver an ascorbic acid derivative into the skin more; thus, an effective action such as a whitening effect that ascorbic acid possesses can be sufficiently exhibited.

What is claimed is:

1. A percutaneous absorption accelerator comprising combination of diglycerol and a low molecular weight betaine.

2. The percutaneous absorption accelerator according to claim 1, wherein the low molecular weight betaine is trimethylglycine.

3. A composition for external use on skin, comprising a percutaneous absorption accelerator according to claim 1 or 2 and an active component.
